(19) United States
(12) Patent Application Publication
(10) Pub. No.: US 2024/0094152 A1
(43) Pub. Date: Mar. 21, 2024

(54) ESR ASSESSMENT OF ASPHALTENE CONTAINING HYDROCARBON STREAMS TO MONITOR ASPHALTENE CONTROL CHEMICAL APPLICATION PERFORMANCE

(71) Applicant: ChampionX LLC, Sugar Land, TX (US)

(72) Inventors: Christopher Alexander Russell, Sugar Land, TX (US); Pritesh Shivshankar Sharma, Missouri City, TX (US); Andrew Robert Neilson, Richmond, TX (US)

(21) Appl. No.: 18/470,695

(22) Filed: Sep. 20, 2023

Related U.S. Application Data
(60) Provisional application No. 63/376,396, filed on Sep. 20, 2022.

(51) Int. Cl. G01N 24/10; G01N 33/28

(52) U.S. Cl. CPC G01N 24/10; G01N 33/2823

(57) ABSTRACT

Methods for assessing the stability of asphaltene by measuring the field strength of the production fluid of asphaltene containing hydrocarbon stream have been developed. These methods can be used for monitoring of asphaltene control chemical application effectiveness during crude oil production and refinement.

### CROSS REFERENCE TO RELATED APPLICATIONS

This patent application claims the benefit of U.S. Provisional Patent Application No. 63/376,396, filed Sep. 20, 2022; the entire disclosure of which is incorporated herein by reference.

### FIELD OF THE INVENTION

Methods for assessing the stability of asphaltene by measuring the field strength of the production fluid of asphaltene containing hydrocarbon stream have been developed. These methods can be used for monitoring of asphaltene control chemical application effectiveness during crude oil production and refinement.

### BACKGROUND OF THE INVENTION

Crude oils include the solubility fractions of maltenes and asphaltenes. Maltenes constitute the fraction of oil that is soluble in low molecular mass n-alkane solvents, such as n-pentane, n-hexane, and n-heptane. Asphaltenes are defined as the crude oil fraction that is soluble in aromatic solvents and insoluble in low-boiling straight chain alkanes. Asphaltene molecules have complex structures and are typically polar molecules with relatively high molecular weights (approximately 700 to 1,000 g/mole). Asphaltenes can contain carbon, hydrogen, nitrogen, oxygen, and sulfur, as well as trace amounts of vanadium and nickel.

Asphaltenes are typically stable under original reservoir conditions, but can be destabilized and precipitate from crude oil during production due to changes in temperature, pressure, chemical composition, and shear rate. Asphaltene deposits can occur throughout the production system, from inside the reservoir formation to pumps, tubing, wellheads, safety valves, flow lines, and surface facilities used in the extraction process. Asphaltene deposits can cause production rate decline and other operational problems, such as increased fluid viscosity and density, and stabilization of oil-water emulsions. The nature of asphaltene deposits, which can appear hard and coal-like or sticky and tar-like, is determined by the composition of the crude oil and the conditions under which precipitation occurred.

Current remediation technologies for asphaltene deposits in the oilfield environment can involve physical and chemical aspects. The deposit, blockage, or obstruction, can be moved by physical force. However, this is often a very expensive operation, and can cause significant loss in production. Chemical methods are relatively less time and cost prohibitive, and typically involve a solvent soak coupled with the addition of an active component to mobilize and solubilize, thereby allowing removal of the deposit. Thus, chemical treatment with additives such as dispersants and inhibitors is one of the commonly adopted control options for the remediation and prevention of asphaltene deposition.

However, although the chemical treatment strategy is frequently used, monitoring of asphaltene control chemical application effectiveness during crude oil production is one of the biggest challenges in the industry. Real time monitoring of asphaltene control chemical effectiveness during crude oil production in the oilfield industry is very challenging and there are very few techniques available to do so. Most techniques (Turbiscan, ADT etc.) requires dilution of crude oil to assess stability of asphaltene in the production fluid. Thus, an improved method for assessing the stability of asphaltene in a production fluid is still needed.

### BRIEF SUMMARY OF THE INVENTION

Various methods are disclosed herein including a method for determining a stability of an asphaltene dispersion.

One aspect of the disclosure is a method of monitoring asphaltene stability in an asphaltene containing hydrocarbon stream using electron spin resonance (ESR) spectroscopy, the method comprising: obtaining an ESR spectrum on the asphaltene containing hydrocarbon stream to measure a field strength of the asphaltene containing hydrocarbon stream and obtain a field strength value; comparing the field strength value of the asphaltene containing hydrocarbon stream with a field strength value of an otherwise identical asphaltene containing hydrocarbon stream tested at a different time; and determining a relative asphaltene stability of the compared asphaltene containing hydrocarbon streams, wherein a higher field strength indicates increased asphaltene stability in the asphaltene containing hydrocarbon stream.

Another aspect of the disclosure is a method of determining effectiveness of an asphaltene control chemical (ACC) agent injected into a asphaltene containing hydrocarbon stream using electron spin resonance (ESR) spectroscopy, the method comprising: obtaining an ESR spectrum on the asphaltene containing hydrocarbon stream to measure a field strength of the asphaltene containing hydrocarbon stream and obtain a field strength value before adding an ACC agent; injecting an amount of the ACC agent into the asphaltene containing hydrocarbon stream to form an ACC agent-treated asphaltene containing hydrocarbon stream; obtaining an ESR spectrum on the ACC agent-treated asphaltene containing hydrocarbon stream to measure a field strength of the ACC agent-treated asphaltene containing hydrocarbon stream and obtain a field strength value; comparing the field strength value of the asphaltene containing hydrocarbon stream with the field strength value of the ACC agent-treated asphaltene containing hydrocarbon stream; and determining the effectiveness of the ACC agent wherein an increase in the field strength value of the ACC agent-treated asphaltene containing hydrocarbon stream compared to the field strength value of the asphaltene containing hydrocarbon stream indicates effectiveness of the asphaltene control chemical.

Yet another aspect of the disclosure is a method of monitoring effectiveness of an asphaltene control chemical (ACC) agent injected into a asphaltene containing hydrocarbon stream using electron spin resonance (ESR) spectroscopy, the method comprising: obtaining an ESR spectrum on a ACC agent-treated asphaltene containing hydrocarbon stream to measure a field strength of the ACC agent-treated asphaltene containing hydrocarbon stream and obtain a first field strength value; obtaining another ESR spectrum on a ACC agent-treated asphaltene containing hydrocarbon stream to measure a field strength of the ACC agent-treated asphaltene containing hydrocarbon stream and obtain a second field strength value after a time interval; and determining the effectiveness of the ACC agent by comparing the second field strength value with the first field strength value, wherein an increase or no change in the second field strength value compared to the first field strength value indicates effectiveness of the asphaltene control chemical.

Other objects and features will be in part apparent and in part pointed out hereinafter.

### BRIEF DESCRIPTION OF THE SEVERAL VIEWS OF THE DRAWING

FIG. 1 depicts example ESR results plot for three example crude oils.

FIG. 2 depicts Relationship of asphaltene content to peak intensity and field strength.

FIG. 3A depicts the correlation between asphaltene concentration via peak area and asphaltene solubility via field strength with heptane dilution of crude oil.

FIG. 3B depicts detection of asphaltene concentration via peak area and transmittance with heptane dilution of crude oil.

FIG. 3C depicts a change in field strength close to flocculation point via peak area and transmittance with heptane dilution of crude oil.

FIG. 4 depicts correlation of change in field strength to separability number/final transmission value with changing dose rate of asphaltene control chemical ASPH17542SP in Asset 1 oil. The labels on the data points correspond to chemical dosage in ppm. Data was generated with aged samples analyzed in Sugar Land RD&E laboratories.

FIG. 5 depicts an ESR plot for Asset 2 crude oil.

FIG. 6 depicts an ESR plot for Asset 3 crude oil.

FIG. 7 depicts an ESR plot for Asset 2 crude oil.

FIG. 8 depicts an ESR plot for field treated Asset 2 crude oil analyzed in the field.

Corresponding reference characters indicate corresponding parts throughout the drawings.

### DETAILED DESCRIPTION OF THE INVENTION

Deposition of various solids from oil field fluids during production can cause wide ranging flow assurance issues. These issues can have significant economic and safety implications. One common deposit type is an asphaltene material, which is a class of crude oil compounds defined by their solubility. It is difficult to determine an effective agent for solvating the asphaltenes in various crude oils. Thus, the methods described herein provide a method for monitoring of asphaltene control chemical application effectiveness during crude oil production. The methods described herein do not require any additional steps (sample preparation, sample dilution) and can be used directly in a production fluid stream.

The quantity of solids deposited on production system structures can be inferred by electron paramagnetic resonance (EPR) techniques even in cases where the pertinent fluids do not themselves exhibit paramagnetism in their natural state. When the fluids become too opaque, other optical techniques require samples to be cleaned up by allowing for settling of solids, heating, and/or centrifuging fluids. In contrast, the EPR systems and methods described herein can avoid such complications. Moreover, the measurements and data collection disclosed herein can be performed real-time and on-site, thereby providing a significant advantage over many other methods.

Electron spin resonance (ESR) spectroscopy is a complex analytical method that uses the energy given off by excited electrons to analyze molecules with unpaired electrons, particularly metal complexes or organic radicals, which makes the method amenable to the analysis of crude oil asphaltenes. Asphaltenes are defined by having a particular solubility and are known to contain metal complexes (e.g., nickel and vanadium) and a relatively high concentration of organic radicals. The intensity of the organic radical peak can be used to examine asphaltene content. However, in the methods described herein, field strength is used to gauge asphaltene stability.

Monitoring of asphaltene control chemical application effectiveness during crude oil production is a significant challenge in the industry. In this disclosure, the stability of asphaltene was assessed by measuring the field strength of the production fluid. Lab experiments showed that the field strength of the production fluid is associated with the stability of the asphaltenes that is a consequence of both the molecular nature of the asphaltene itself and the solvency power of the surrounding crude oil medium. Thus, the method allows assessment of asphaltene control chemical application effectiveness during crude oil production using an online monitoring system.

In the methods described herein, the stability of asphaltenes was assessed by measuring the field strength of the production fluid.

Various methods are disclosed herein including a method of monitoring asphaltene stability in an asphaltene containing hydrocarbon stream using electron spin resonance (ESR) spectroscopy, the method comprising: obtaining an ESR spectrum on the asphaltene containing hydrocarbon stream to measure a field strength of the asphaltene containing hydrocarbon stream and obtain a field strength value; comparing the field strength value of the asphaltene containing hydrocarbon stream with a field strength value of an otherwise identical asphaltene containing hydrocarbon stream tested at a different time; and determining a relative asphaltene stability of the compared asphaltene containing hydrocarbon streams, wherein a higher field strength indicates increased asphaltene stability in the asphaltene containing hydrocarbon stream.

The method can further comprise measuring a field strength of the asphaltene containing hydrocarbon stream after a time interval to determine changes in asphaltene stability over time. The time interval can be 15 minutes, 30 minutes, 45 minutes, 1 hour, 5 hours, 10 hours, 15 hours, 20 hours, or 1 day.

The ESR spectroscopy test can be performed on the asphaltene containing hydrocarbon stream without additional sample preparation.

The disclosure is also directed to a method of determining effectiveness of an asphaltene control chemical (ACC) agent injected into an asphaltene containing hydrocarbon stream using electron spin resonance (ESR) spectroscopy, the method comprising: obtaining an ESR spectrum on the asphaltene containing hydrocarbon stream to measure a field strength of the asphaltene containing hydrocarbon stream and obtain a field strength value before adding an ACC agent; injecting an amount of the ACC agent into the asphaltene containing hydrocarbon stream to form an ACC agent-treated asphaltene containing hydrocarbon stream; obtaining an ESR spectrum on the ACC agent-treated asphaltene containing hydrocarbon stream to measure a field strength of the ACC agent-treated asphaltene containing hydrocarbon stream and obtain a field strength value; comparing the field strength value of the asphaltene containing hydrocarbon stream with the field strength value of the ACC agent-treated asphaltene containing hydrocarbon stream; and determining the effectiveness of the ACC agent wherein an increase in the field strength value of the ACC agent-treated asphaltene containing hydrocarbon stream compared to the field strength value of the asphaltene containing hydrocarbon stream indicates effectiveness of the asphaltene control chemical.

The disclosure is further directed to a method of monitoring effectiveness of an asphaltene control chemical (ACC) agent injected into a asphaltene containing hydrocarbon stream using electron spin resonance (ESR) spectroscopy, the method comprising: obtaining an ESR spectrum on a ACC agent-treated asphaltene containing hydrocarbon stream to measure a field strength of the ACC agent-treated asphaltene containing hydrocarbon stream and obtain a first field strength value; obtaining another ESR spectrum on a ACC agent-treated asphaltene containing hydrocarbon stream to measure a field strength of the ACC agent-treated asphaltene containing hydrocarbon stream and obtain a second field strength value after a time interval; and determining the effectiveness of the ACC agent by comparing the second field strength value with the first field strength value, wherein an increase or no change in the second field strength value compared to the first field strength value indicates effectiveness of the asphaltene control chemical.

The method can further comprise modifying the identity of the ACC agent, the amount of the ACC agent, or both the identity and amount of the ACC agent injected into the production stream based on the determined effectiveness of the ACC agent; and obtaining another ESR spectrum on a ACC agent-treated asphaltene containing hydrocarbon stream to measure a field strength of the ACC agent-treated asphaltene containing hydrocarbon stream and obtain a third field strength value.

The identity and/or amount of the ACC agent can be modified. For example, either a different ACC agent or an increased amount of the ACC agent is injected into the asphaltene containing hydrocarbon stream if the second field strength value is decreased as compared to the first field strength value.

For the methods, the ACC agents can be selected from the group consisting of diethylene triamine (DETA)/tall oil fatty acid (TOFA)-imidazoline, DETA/TOFA-imidazoline acrylate, DETA/TOFA-imidazolinium, DETA/TOFA-imidazolinium acrylate, or a combination thereof.

The ACC agents can comprise a charged surfactant and/or a polymer. The charged surfactant can be nonpolymeric. The charged surfactant can also be an anionic surfactant, a cationic surfactant, an amphoteric surfactant, a zwitterionic surfactant, or a combination thereof.

The polymer can be nonionic. The polymer can have an average molecular weight ranging from about 500 Da to about 500,000 Da. The polymer can comprise a monomer selected from the group consisting of isobutylene, butadiene, isoprene, ethylene, propylene, an acrylate, acrylamide, methacrylate, methacrylamide, or a combination thereof. The polymer can comprise an alkyl phenol-formaldehyde polymer, an alkyl phenol-amine-formaldehyde polymer, a polyalkylene, a polyisobutylene succinic ester, or a polyisobutylene succinimide. The polymer can comprise about 25 mole % to about 75 mole % of a nonylphenol and about 25 mole % to about 75 mole % of formaldehyde. The polymer can comprise a polyisobutylene succinic anhydride pentaerythritol ester, a dinonylphenol-formaldehyde-nonylphenol polymer, or a 4-nonylphenol-formaldehyde-diethylenetriamine.

The ACC agent can be added to the asphaltene containing hydrocarbon stream at a dosage of from about 1 ppm to about 5000 ppm, from about 1 ppm to about 4000 ppm, from about 1 ppm to about 3000 ppm, from about 1 ppm to about 2000 ppm, from about 1 ppm to about 1000 ppm, from about 10 ppm to about 5000 ppm, from about 10 ppm to about 4000 ppm, from about 10 ppm to about 3000 ppm, from about 10 ppm to about 2000 ppm, from about 10 ppm to about 1000 ppm, from about 50 ppm to about 5000 ppm, from about 50 ppm to about 4000 ppm, from about 50 ppm to about 3000 ppm, from about 50 ppm to about 2000 ppm, from about 50 ppm to about 1000 ppm, from about 100 ppm to about 5000 ppm, from about 100 ppm to about 4000 ppm, from about 100 ppm to about 3000 ppm, from about 100 ppm to about 2000 ppm, from about 100 ppm to about 1000 ppm, based on the total volume of the asphaltene containing hydrocarbon stream. Preferably, the ACC agent is added to the asphaltene containing hydrocarbon stream at a dosage of from about 100 ppm to about 1000 ppm, based on the total volume of the asphaltene containing hydrocarbon stream.

The asphaltene containing hydrocarbon stream can comprise any asphaltene containing hydrocarbon stream where stability control is needed. Preferably, the asphaltene containing hydrocarbon stream is crude oil or a refined crude oil.

The methods of this disclosure can be implemented in an oil field, in an oil refinery, or in an oil sands upgrading facility.

Any of various species in the fluid may be measured by the EPR sensor. For example, the species being sensed may include free radical and transition metal ions, such as asphaltene (free radicals), scales, spin probes, inhibitors, and/or ions, that have an EPR signature. The EPR sensor may provide a spectroscopic view of the paramagnetic components of the sample.

The EPR sensor may be disposed at any of various suitable locations (e.g., to implement the operations described herein). For example, the EPR sensor may be located downhole, at a wellhead producer (i.e., the wellhead of a production well), at a wellhead injector (i.e., the wellhead of an injection well), at a header or gathering facility, at a test separator or facility, at a storage, at an input to a refinery, or in the refinery process. In this manner, the species of interest may be continuously monitored throughout a field or a process, at one or more locations as desired. Furthermore, the system can be adjusted in real-time based on the characteristics of the species measured with the EPR sensor(s). As an example, paramagnetic sensing downstream of an interaction may be used to guide upstream injection.

For example, the EPR sensor may be positioned at a wellhead (e.g., of an injection well or a production well) to measure the asphaltene solubility and the asphaltene stabililty. Chemicals (e.g., asphaltene inhibitors) can be injected into the well. The EPR sensor allows for measurements of the resulting fluid, so the amount of inhibitors being injected may be adjusted accordingly. For example, if an insufficient amount of the asphaltene inhibitor is being injected, the EPR sensor can measure a decrease in the asphaltene solubility and stability in real time, and the asphaltene inhibitor injection can be increased based on these measurements.

In some embodiments, a portable EPR device may be in fluid communication with a wellhead, oilfield tubular, or downhole device and may detect the presence and effect of an asphaltene inhibitor without human intervention to take a sample. The feedback from the portable EPR device can be used in an automatic control system for use with automated injection of asphaltene inhibitor (e.g., to maintain asphaltene solubility and stability). The methods described herein can include monitoring (periodically and/or continuously) a fluid using an EPR device and periodically (e.g., once a month) making an additional measurement.

As used herein, the term “asphaltene” refers to a class of hydrocarbons in carbonaceous material, such as crude oil, bitumen, or coal that is soluble in toluene, xylene, and benzene, yet insoluble in n-alkanes, e.g., n-heptane and n-pentane. Asphaltenes are generally characterized by fused ring aromaticity with some small aliphatic side chains, and typically some polar heteroatom-containing functional groups, e.g., carboxylic acids, carbonyl, phenol, pyrroles, and pyridines, capable of donating or accepting protons intermolecularly and/or intramolecularly, having a molar H/C ratio of about 1 to 1.2, and a N, S, and O content of a low weight percent.

As used herein, the term “electron spin resonance spectroscopy” or “ESR spectrosphy” refers to a complex analytical method that uses the energy given off by excited electrons to analyze molecules with unpaired electrons, particularly metal complexes or organic radicals, which makes the method amenable to the analysis of crude oil asphaltenes.

As used herein, the term “precipitation propensity” refers to the tendency of a composition that includes a first crude oil or a composition that includes a first crude oil and at least one second crude oil to precipitate asphaltenes, where at least one of the first crude oil and the second crude oil includes asphaltenes. The precipitation propensity can be measured by any conventional technique for measuring asphaltene precipitation or aggregation, including, but not limited to, volumetric solvent titrimetry with optical measurement, e.g., infrared spectroscopy and/or near infrared spectroscopy, including oil compatibility models; Asphaltene Stability Index (ASI) test using solvent-titration, as described in Gawrey, et al., Instrumentation Science & Technology, 2004, 32(3), 247-253; solvent titrimetry with electrical measurement, e.g., conductivity and/or capacitance, as described in U.S. Pat. No.: 5,420,040; solvent titrimetry with surface tension measurement, as described in U.S. Pat. No. 5,420,040; spot testing, as described in ASTM E 4740 (2004); viscometry, as described in J. Escobedo, et al., “Viscometric Determination of the Onset of Asphaltene Flocculation: A Novel Method,” Society of Petroleum Engineers, May 1995; optical microscopy; refractive indices measurement, as described in ASTM E 1218 (2012); vapor pressure osmometry, as described in U.S. Pat. No. 5,420,040 and Gawrey, et al., Instrumentation Science & Technology, 2004, 32(3), 247-253); gravimetric titrimetry, as described in U.S. Pat. No. 5,420,040; autoclaving; colloidal instability index, as described in Gawrey, et al., Instrumentation Science & Technology, 2004, 32(3), 247-253; detection of bubble points and asphaltene aggregation onset pressures by NIR, as described by Aske, et al., Energy & Fuels, 2002, 16, 1287-1295; nuclear magnetic resonance (NMR) relaxometry, as described in Prunelet et al., C. R. Chimie 7 (2004); pulsed-field gradient spin echo nuclear magnetic resonance (NMR), as described in Gawrey, et al., Instrumentation Science & Technology, 2004, 32(3), 247-253; small-angle neutron scattering, as described in Gawrey, et al., Instrumentation Science & Technology, 2004, 32(3), 247-253; saturates, asphaltenes, resins, aromatics (SARA) analysis, where NR>0.35 is unstable, as described in Falkler et al., Hydrocarbon Processing, September 2010, 67-73; or a combination thereof.

As used herein, the term “solubility ratio” refers to a precipitation propensity of a crude oil determined by: (i) adding an asphaltene non-solvent to an initial volume of a crude oil; (ii) measuring the volume of the asphaltene non-solvent that causes asphaltene precipitation, e.g., by determining a minimum optical density as measured by near infrared spectroscopy; and (iii) dividing the volume of the asphaltene non-solvent added to the crude oil by the initial volume of the crude oil.

As used herein, the term “near-infrared spectrometry” or “NIR spectrometry” refers to spectroscopic methods that use the near-infrared region of the electromagnetic spectrum from about 800 nm to about 2,500 nm. The methods discussed and described herein can be used to predict the compatibility of a wide range of different crude oils before blending, including operable proportions, to reduce, minimize, prevent, or eliminate asphaltene precipitation and/or one or more of the problems caused by asphaltene precipitation such as unplanned refinery events caused by asphaltene precipitation. The proportions of any number of crude oils in a blend and/or to be combined with one another to form a blend can also be determined to help optimize crude rate and/or crude blend compatibility. The methods and the associated calculations allow a precipitation propensity, such as a solubility ratio as measured by near-infrared spectrometry, to be determined for the crude oil blend components.

As used herein, the term “optical density” (i.e., OD) refers to the attenuated measurement in the incident light due to absorbance and scattering by a medium through which the light travels. Asphaltenes can flocculate from solution with the addition of an asphaltene non-solvent, e.g., an n-alkane such as n-heptane, and the OD can be affected by the flocculation of the asphaltenes. The optical density at a wavelength of about 1,600 nm can be of particular interest since it corresponds to a region associated with relatively low background absorbance for crude oil. As asphaltene nonsolvent is added to a crude oil sample (also referred to as “titration”), the NIR absorbance and the OD at about 1,600 nm decrease initially due to dilution of the sample; however, asphaltene flocculation causes an increase in the OD as the transmitted light is reduced due to the scattering and absorbance of light by the flocculated asphaltenes. Hence, a minimum is observed in the OD at about 1,600 nm with addition of an asphaltene non-solvent such as n-heptane. It is the volume of asphaltene non-solvent corresponding to the minimum OD that is reported as the onset point of asphaltene flocculation and used in the solubility ratio.

Having described the invention in detail, it will be apparent that modifications and variations are possible without departing from the scope of the invention defined herein.

### EXAMPLES

The following non-limiting examples are provided to further illustrate the present disclosure.

### Example 1: ESR Analysis

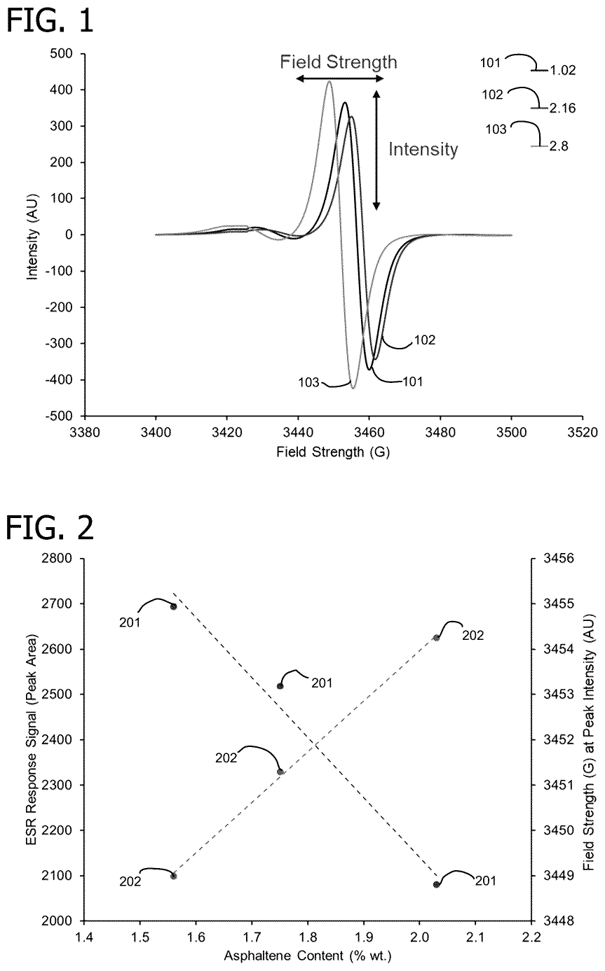

FIG. 1 displays typical results from ESR analysis of example crude oils. Reference characters 101, 102, 103 relate the plot to the legend. The large peak is the organic radical, and the intensity of this peak (height or area) can be correlated to the asphaltene content of the crude oil. The apex of this peak can also vary in position along the x-axis, which is the field strength, or G-Factor, which is thought to be related to the solubility of the asphaltenes that is a consequence of both the molecular nature of the asphaltene itself and the solvency power of the surrounding crude oil medium.

The data displayed in FIG. 2 demonstrate the relationship of asphaltene content to both organic radical peak intensity and field strength. As the asphaltene content increases, the organic radical peak intensity increases. Concurrently, there is a reduction in the field strength (points 201) at the highest peak intensity (points 202). The data indicates that it may be possible to determine asphaltene content and solubility characteristics using the ESR. However, for product performance monitoring, it may be more important to examine overall solubility as it is believed that enhanced solubility of asphaltenes in the crude oil medium is one mechanism of effective asphaltene control chemical performance.

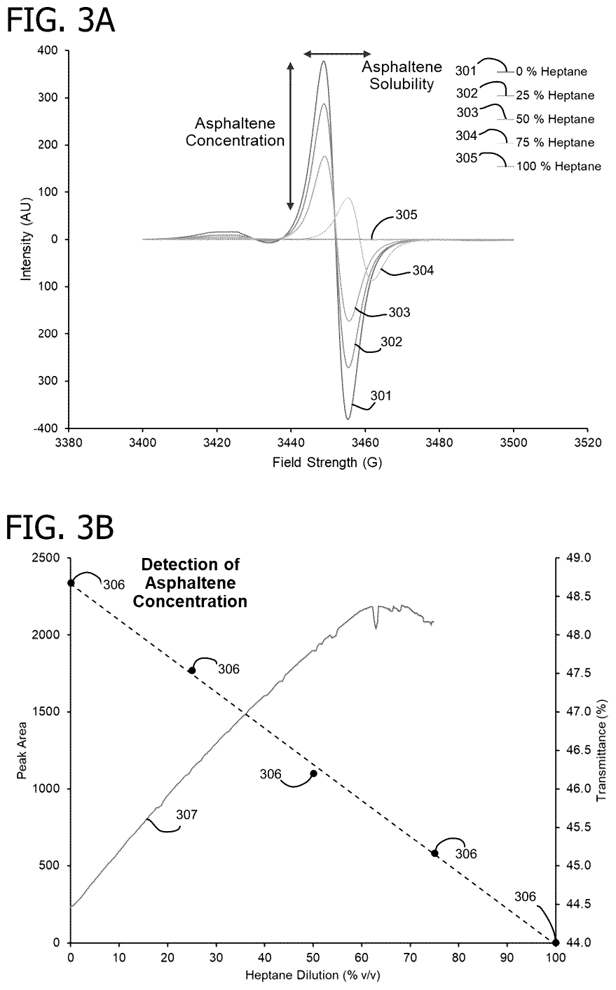

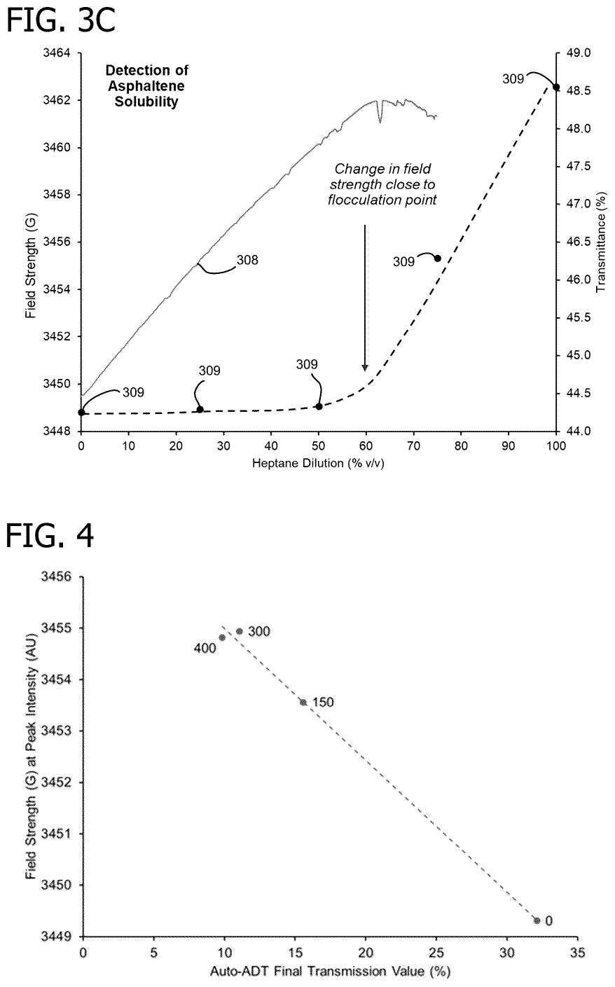

Asphaltene solubility within the crude oil was changed by sequentially adding heptane and measuring the peak intensities and field strength via ESR. The data is displayed in FIG. 3.

FIG. 3A shows how the ESR response changes with the addition of heptane to the crude oil. Reference characters 301, 302, 303, 304, 305 relate the plot to the legend. The intensity of the peak is changing dramatically with each addition, but the location on the x-axis (field strength) is not for up to 50% heptane. This is a consequence of dilution and is exemplified in FIG. 3B by the points 306 corresponding to the primary y-axis. Also plotted in FIG. 3B, using the secondary y-axis, is the titration profile of the crude oil (plot 307) as measured by NIR transmittance, also known as the flocculation point profile. Indeed, the flocculation point is observed between 60 and 70% v/v heptane explaining the change in field strength for the 75% heptane result in FIG. 3A. The flocculation point profile is again displayed in FIG. 3C (plot 308), but here with the field strength as the points 309 on the primary y-axis. This value remains constant until the flocculation point is reached, after which a large increase is observed.

The data suggests that changes in the nature of the asphaltenes as they are aggregating, other than concentration, i.e. changes due to asphaltene control chemical application, are detectable by the change in field strength.

### Example 2: ESR Assessment Completed in the Lab (For Field and Lab Treated Samples)

Field treated samples from Asset 1 were delivered to the RD&E laboratories to investigate the possibility of using ESR to detect positive effects of asphaltene control chemical treatment for use as a possible monitoring technique, for example. The ESR readings were compared to the assessment of asphaltene relative stability as performed in the field using separability number via Turbiscan experiments. The comparison is displayed in FIG. 4. Significantly, samples that were untreated and had a relatively large separability number (high final transmission value on the x-axis) revealed a relatively low field strength value. As dosage was increased, the separability number decreased as the field strength increased. The linear correlation as displayed on FIG. 4 is remarkably good. It was then decided to perform a larger field treated crude oil ESR testing campaign.

For the analysis, the oil sample is loaded into a test cell, inserted into the machine, an analyze button depressed, and a spectrum is generated.

Table 1 and Table 2 outline the information as it pertains to ESR assessment of field treated crude oils from different locations to monitor asphaltene control chemical application performance for cases 1-3. Asphaltene control chemical (ACC) Al is a mixture of phenolic resin and surfactants.

TABLE 1
ESR Assessment of Field Treated Crude Oils

| Case | Location | ACC | Dosage |
|---|---|---|---|
| 1 | Asset 2 | A1 | 0-1000 ppm |
| 2 | Asset 3 | A1 | 0-500 ppm |
| 3 | Asset 4 | A1 | 150-750 ppm |

TABLE 2
ESR Assessment of Lab Treated Crude Oils

| Case | Location | AI | Dosage |
|---|---|---|---|
| 4 | Asset 5 | Various | 500 ppm |
| 5 | Asset 6 | Various | 0-250 ppm |
| 6 | Asset 7 | Various | 0-250 ppm |

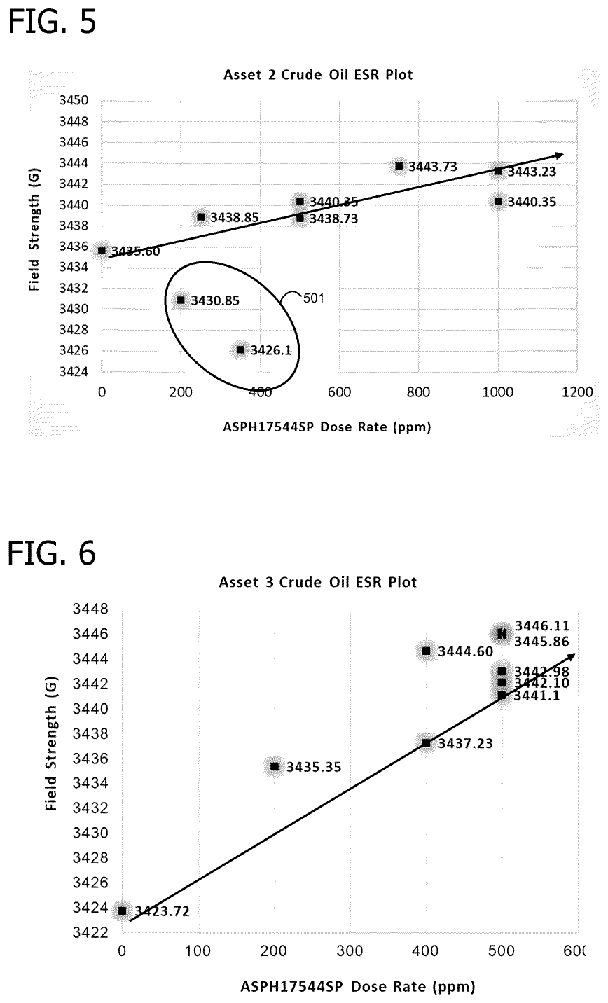

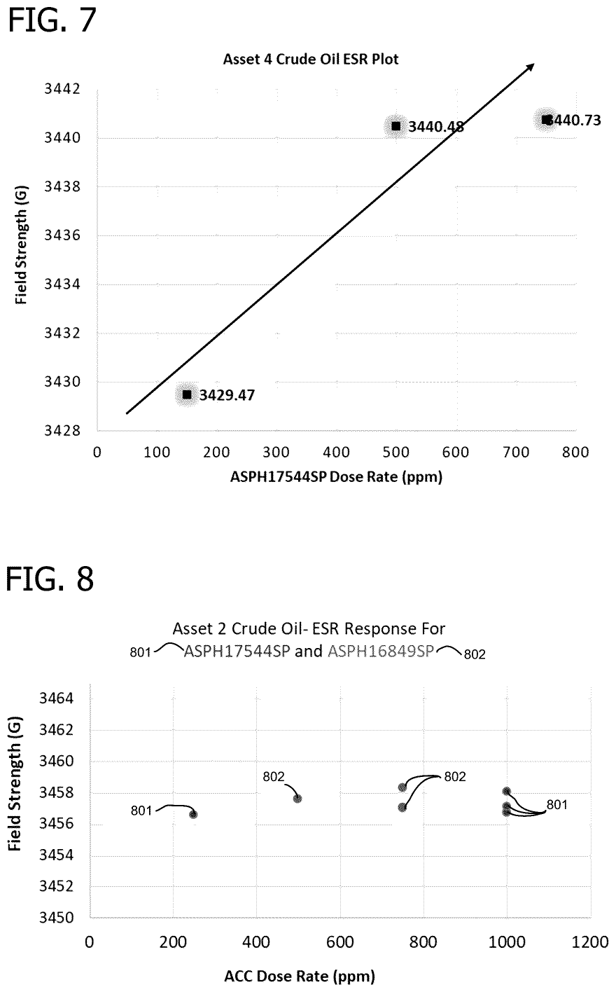

Case 1-3 (Tables 3-5, FIGS. 5-7) summarizes results of ESR assessment completed in the lab for field treated crude oil samples, i.e., crude oils samples collected in the field as ACC treatment was in progress, subsea. Overall, a good correlation was observed for magnetic field strength value for most field treated samples as analyzed in the Sugar Land laboratory, with only two potential outliers identified (oval 501 in FIG. 5). Most ACC treated crude oil samples received from the field showed a consistent trend of higher magnetic field strength value with increasing dose rate.

### Case 1: ESR Assessment of Asset 2 Field Treated Sample Using A1

TABLE 3
ESR Assessment Data for Field Treated
Asset 2 Crude Oil Analyzed in the Lab

| | Dose Rate | Magnetic Field Strength |
|---|---|---|
| Asphaltene Control Chemical | (ppm) | (G) |
| — | 0 | 3435.60 |
| A1 | 1000 | 3443.23 |
| | 1000 | 3440.35 |
| | 750 | 3443.73 |
| | 500 | 3438.73 |
| | 500 | 3440.35 |
| | 350 | 3426.1 |
| | 250 | 3438.85 |
| | 200 | 3430.85 |

### Case 2: ESR Assessment of Asset 3 Field Treated Sample Using A1

TABLE 4
ESR Assessment Data for Field Treated
Asset 3 Crude Oil Analyzed in the Lab

| Asphaltene Control | Dose Rate | Magnetic Field Strength |
|---|---|---|
| Chemical | (ppm) | (G) |
| — | 0 | 3423.72 |
| A1 | 500 | 3445.86 |
| | 500 | 3442.98 |
| | 500 | 3442.10 |
| | 500 | 3446.11 |
| | 400 | 3444.60 |
| | 400 | 3437.23 |
| | 500 | 3441.1 |
| | 200 | 3435.35 |

### Case 3: ESR Assessment of Asset 4 Field Treated Sample Using A1

TABLE 5
ESR Assessment Data for Field Asset
4 Crude Oil Analyzed in the Lab

| Asphaltene Control | Dose Rate | Magnetic Field Strength |
|---|---|---|
| Chemical | (ppm) | (G) |
| A1 | 150 | 3429.47 |
| | 500 | 3440.48 |
| | 750 | 3440.73 |

Overall, good correlation was observed between magnetic field strength value and asphaltene control chemical (ACC) dose rate for many field treated samples when they were evaluated in the Sugar Land RD&E lab several weeks following field treatment (Cases 1-3).

FIG. 8 depicts an ESR plot for field treated Asset 2 crude oil analyzed in the field, wherein reference characters 801, 802 relate the plot to the legend.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

1. A method of monitoring asphaltene stability in an asphaltene containing hydrocarbon stream using electron spin resonance (ESR) spectroscopy, the method comprising:
obtaining an ESR spectrum on the asphaltene containing hydrocarbon stream to measure a field strength of the asphaltene containing hydrocarbon stream and obtain a field strength value;
comparing the field strength value of the asphaltene containing hydrocarbon stream with a field strength value of an otherwise identical asphaltene containing hydrocarbon stream tested at a different time; and
determining a relative asphaltene stability of the compared asphaltene containing hydrocarbon streams, wherein a higher field strength indicates increased asphaltene stability in the asphaltene containing hydrocarbon stream.

2. The method of claim 1, further comprising measuring a field strength of the asphaltene containing hydrocarbon stream after a time interval to determine changes in asphaltene stability over time.

3. The method of claim 2, wherein the time interval is 15 minutes, 1 hour, or 1 day.

4. The method of any one of claims claim 1 to-3, wherein the ESR spectroscopy test is performed on the asphaltene containing hydrocarbon stream without additional sample preparation.

5. A method of determining effectiveness of an asphaltene control chemical (ACC) agent injected into a asphaltene containing hydrocarbon stream using electron spin resonance (ESR) spectroscopy, the method comprising:
obtaining an ESR spectrum on the asphaltene containing hydrocarbon stream to measure a field strength of the asphaltene containing hydrocarbon stream and obtain a field strength value before adding an ACC agent;
injecting an amount of the ACC agent into the asphaltene containing hydrocarbon stream to form an ACC agent-treated asphaltene containing hydrocarbon stream;
obtaining an ESR spectrum on the ACC agent-treated asphaltene containing hydrocarbon stream to measure a field strength of the ACC agent-treated asphaltene containing hydrocarbon stream and obtain a field strength value;
comparing the field strength value of the asphaltene containing hydrocarbon stream with the field strength value of the ACC agent-treated asphaltene containing hydrocarbon stream; and
determining the effectiveness of the ACC agent wherein an increase in the field strength value of the ACC agent-treated asphaltene containing hydrocarbon stream compared to the field strength value of the asphaltene containing hydrocarbon stream indicates effectiveness of the asphaltene control chemical.

6. A method of monitoring effectiveness of an asphaltene control chemical (ACC) agent injected into a asphaltene containing hydrocarbon stream using electron spin resonance (ESR) spectroscopy, the method comprising:
obtaining an ESR spectrum on a ACC agent-treated asphaltene containing hydrocarbon stream to measure a field strength of the ACC agent-treated asphaltene containing hydrocarbon stream and obtain a first field strength value;
obtaining another ESR spectrum on a ACC agent-treated asphaltene containing hydrocarbon stream to measure a field strength of the ACC agent-treated asphaltene containing hydrocarbon stream and obtain a second field strength value after a time interval; and
determining the effectiveness of the ACC agent by comparing the second field strength value with the first field strength value, wherein an increase or no change in the second field strength value compared to the first field strength value indicates effectiveness of the asphaltene control chemical.

7. The method of claim 6, further comprising modifying the identity of the ACC agent, the amount of the ACC agent, or both the identity and amount of the ACC agent injected into the production stream based on the determined effectiveness of the ACC agent; and obtaining another ESR spectrum on a ACC agent-treated asphaltene containing hydrocarbon stream to measure a field strength of the ACC agent-treated asphaltene containing hydrocarbon stream and obtain a third field strength value.

8. The method of claim 6, wherein the time interval is 1 day.

9. The method of claim 7, wherein the identity of the ACC agent is modified.

10. The method of claim 7, wherein the amount of the ACC agent is modified.

11. The method of claim 7, wherein both the identity and amount of the ACC agent are modified.

12. The method of claim 6, wherein either a different ACC agent or an increased amount of the ACC agent is injected into the asphaltene containing hydrocarbon stream if the second field strength value is decreased as compared to the first field strength value.

13. The method of claim 6, wherein the ACC agent(s) is(are) selected from the group consisting of diethylene triamine (DETA)/tall oil fatty acid (TOFA)-imidazoline, DETA/TOFA-imidazoline acrylate, DETA/TOFA-imidazolinium, DETA/TOFA-imidazolinium acrylate, or a combination thereof.

14. The method of claims 6, wherein the ACC agent(s) comprise(s) a charged surfactant and/or a polymer.

15. The method of claim 14, wherein the charged surfactant is nonpolymeric.

16. (canceled)

17. The method of claim 14, wherein the polymer is nonionic.

18.-19. (canceled)

20. The method of claim 14, wherein the polymer comprises an alkyl phenol-formaldehyde polymer, an alkyl phenol-amine-formaldehyde polymer, a polyalkylene, a polyisobutylene succinic ester, or a polyisobutylene succinimide.

21.-22. (canceled)

23. The method of claim 6, wherein the ACC agent is added to the asphaltene containing hydrocarbon stream at a dosage of from about 1 ppm to about 2000 ppm, based on the total volume of the asphaltene containing hydrocarbon stream.

24. The method of claim 2, wherein the ACC agent is added to the asphaltene containing hydrocarbon stream at a dosage of from about 100 ppm to about 1000 ppm, based on the total volume of the asphaltene containing hydrocarbon stream.

25.-26. (canceled)

27. The method of claim 1, wherein the method is implemented in an oil field, in an oil refinery, or in an oil sands upgrading facility.
